(19) United States
(12) Patent Application Publication
(10) Pub. No.: US 2003/0078392 A1
(43) Pub. Date: Apr. 24, 2003

(54) Milk and cheese modification process, including methods of extracting beta-lactoglobulin and caseins from milk and milk products, and novel products thereby produced

(76) Inventors: Jeff Leaver, Ayr (GB); Andrew Law, Ayr (GB)

Correspondence Address: Fleshner & Kim, PO Box 221200, Chantilly, VA, 20153-1200, US

(21) Appl. No.: 10/181,725

(22) PCT Filed: Jan. 22, 2001
(86) PCT No.: PCT/GB01/00208

(30) Foreign Application Priority Data
Jan. 22, 2000 (GB) 00001433.2

(51) Int. Cl. C07K 1/30; C07K 14/47

(52) U.S. Cl. 530/420; 530/380

(57) ABSTRACT

A method for diaggregating and reforming the casein micelles of milk to produce a product with physical properties differing significantly from that of the original milk. There is also provided a milk fraction highly enriched in beta-lactoglobulin (BLG) and a soluble whey fraction correspondingly depleted.

The present invention relates to a novel method for disaggregating and reforming casein micelles in milk by adjusting the pH and also for selectively precipitating a single protein, β-lactoglobulin, from the whey fraction of milk. It relates further to modified milk, cheese and food products and methods for making the same and a process for separating casein fractions using a caseinate feedstock.

The invention relates in particular to a method for disaggregating and reforming the casein micelles of milk to give a product whose physical properties differ significantly from that of the original milk. In addition, a single protein, β-lactoglobulin (BLG), is selectively precipitated from the mixture of soluble proteins present in the whey fraction of milk to yield a fraction which is highly enriched in this protein and a soluble whey fraction which is correspondingly depleted. The method is based on changing the pH of both milk and of acid- and cheese-whey. The BLG is selectively precipitated and can easily be recovered by filtration or centrifugation or alternatively it can be co-precipitated with caseins in caseinate and cheese manufacture.

Milk, especially bovine milk, is a major source of human nutrition. World milk production was estimated to be 557 million tonnes in 1998, an increase of 1.4% on the previous year. Milk itself is a relatively complex mixture of fat, protein, minerals and sugar (lactose). Protein content is in the region of 35 g/L and the proteins in milk can be conveniently divided into two major classes, the whey proteins and the caseins. Approximately 80% of the protein content is packaged, together with much of the calcium and phosphate, in sub-micron-sized colloidal particles which are termed the casein Nacelles. Scattering of light by the casein micelles is the reason why milk appears white. The integrity of the casein micelles is crucial to the stability of milk and the properties of products made from milk are in part determined by the properties of the micelles. For example, it is known that the firmness of cheeses can be directly correlated with the average diameter of the micelles in the milk from which they are made. Worldwide cheese production was estimated to be in the region of 15 million tonnes in 1998. Increasingly, milk proteins are consumed not as liquid milk but as ingredients in products that have been processed to a greater or lesser extent. The degree of processing ranges from yogurt and cottage cheese manufacture, where the micelles are induced to aggregate by acidification, to the incorporation of milk protein fractions into sauces and spreads.

The whey fraction of milk is the liquid portion remaining after the casein micelles have been precipitated, either by addition of proteolytic enzymes in the case of cheese manufacture, or by acidification in the case of caseinate manufacture. Global whey production is estimated at 118 million tonnes, of which 66% is manufactured in Europe and 25% in North America. 92% was generated from cheese production and 8% from caseinate production. The whey fraction contains a number of proteins which are nutritionally of a high quality but which are present in a dilute form together with lactose and inorganic salts. Since the protein content of whey is approximately 8 g/L and 50% of this is BLG, this equates to 940,000 tonnes of protein and 470,000 tonnes of BLG.

Due to the dilute nature of the solution, protein recovery is difficult and relatively expensive. Formerly, whey was treated as a waste by-product from the processing of milk and much was used as animal feed in a liquid form or disposed of directly into water courses. However, due to the high Biological Oxygen Demand (BOD) resulting from its high content of the sugar, lactose, together with the proteins, this latter procedure led to eutrophication of water courses and is now banned. The cost of processing any material through the normal sewerage system must now be borne by the producer. Much of the liquid whey is now processed to enable recovery of the protein and lactose but significant amounts are still used as animal feed. Current recovery processes are generally high energy techniques and/or require expensive, specialised equipment. In general the finished product is a mixture of all of the proteins present in the original whey. A simple, low energy method of recovering BLG from whey would be beneficial since it would yield a valuable by-product at relatively little cost. An aim of the present invention is to provide a method for recovery of protein from whey and an important 8 associated goal of this invention is to provide such of method of BLG preparation which is cheap and low energy.

The present invention yields fractionated whey proteins which may also have properties that are different from those of the unfractionated whey and may therefore be attractive to manufacturers in a number of fields such as the food and pharmaceutical industries. Within the food processing industry, whey proteins are used as for their ability to stabilise emulsions and foams, as fat replacers, to bind water and as gelation agents. In addition BLG can bind hydrophobic ligands and there is some interest within the food industry in using this to bind flavour molecules and within the perfume industry and pharmaceutical industry to bind aromas and hydrophobic drugs.

### Prior Art Techniques for Manufacturing Cheese

As a result of the interest in the manufacture of cheese and yoghurt where the pH of the milk decreases during manufacture as a result of the enzymatic action, a great, deal is known about the effects of acidification on the structure and function of milk. In contrast, very little is known about the effects of alkaline treatment on the properties of milk.

Fundamentally cheese manufacture involves inducing the aggregation of casein micelles either by acidification, or more commonly by the addition of proteolytic enzymes such as rennet which hydrolyse the casein component that stabilises the outer surface of the micelles. In cheeses which undergo a maturation period prior to consumption, other enzymes released from micro-organisms either naturally present in the milk or deliberately added, subsequently perform further hydrolysis of fat, carbohydrates and proteins during the maturation period. Traditionally raw milk is used in the process but due to hygiene considerations, most milk is pasteurised. Incorporation of the BLG fraction into cheese would potentially increase the protein content by approximately 14% and may also influence the characteristics of the final product.

Conventional cheese manufacture uses either unheated or pasteurised milk which has been subjected to mild heating to kill potentially harmful organisms. Aggregation of the micellar proteins is achieved by addition of proteolytic enzymes such as chymosin or acidification to pH 4.6. The aggregate formed is termed the curd and the liquid portion the whey. Approximately 30% of the total protein present in the milk is located in the whey and is lost from the cheese-making process. Attempts have been made to incorporate some of this protein into the curd by heating. However, this requires temperatures in excess of 75° C. for relatively long periods and has detrimental effects on both the processing of the milk and the flavour and texture characteristics of the cheese. An alternative method to incorporate whey proteins into cheese is to add insoluble whey protein powder produced from separated whey back into the curd.

In the manufacture of cottage cheese, milk is acidified by the action of an acid-producing bacteria or by the addition of acid and as the pH reaches 4.6 to 5.0, the micelles precipitate and aggregate. As in the enzyme-induced aggregation, under normal manufacturing conditions no significant amounts of whey proteins are incorporated into the cheeses

### Prior Art Methods for Recovering Whey Proteins and Incorporating them Into Dairy Products

Due to the very dilute nature of proteins within the whey, recovery is difficult and relatively expensive at the present time.

Whey powder is manufactured mainly from sweet whey obtained from cheese making by evaporating clarified whey to 40 to 62% solids followed by spray drying and sometimes a final drying stage in a vibrating fluid bed. The high mineral content of whey powder makes it unsuitable for some applications such as animal feeds and baby formulations Processes exist to reduce the mineral content. These include ion-exchange chromatography in which the pasteurised whey is passed in series through columns packed with anionic and cationic ion-exchange resins. Approximately 90 to 98% of the minerals can be removed. Electrodialysis, involves passing whey concentrated to 20 to 30% solids through an electrodialysis cell consisting of alternating cation- and anion-selective membranes behind which water is recirculated. An electrical current is applied and the ions migrate to the electrodes. Typically 90% demineralisation can be achieved. Nanofiltration, a membrane separation technique, permits monovalent ions to pass through a membrane retaining the proteins and lactose.

Whey proteins may also be recovered by heat precipitation. Typically this involves heating whey, which may have been demineralized and concentrated, at 90 to 95° C. and pH 4.4 to 4.8 for 30 to 50 minutes. The proteins denature and aggregate and are removed by settling, either static or accelerated and the precipitated protein is then washed, reseparated and dried.

Lactose and salts can also be removed from clarified whey by ultrafiltration in which a membrane is used to retain protein producing a whey protein concentrate which, after drying, contains 30 to 80% protein. As a refinement, ultrafiltration membranes with very specific molecular weight cut-off values can be used to achieve fractionation of the proteins to produce relatively pure BLG. However these systems are still relatively small scale and whether the process is financially viable is still to be proved.

Whey proteins can also be recovered by the addition of complexing agents. As an example, long-chain polyphosphates are added to whey at low pH e.g. 2.5. The precipitate so formed is removed by centrifugation, washed and then subjected to pH alteration and calcium addition to remove the phosphate. Up to 90% of the whey protein can be recovered.

Whey protein may also be recovered by ion-exchange chromatography. Protein is adsorbed to suitable ion-exchange resins either packed in columns or in stirred tanks. After removing the deproteinated whey, proteins are deadsorbed by changing the pH and the eluent is ultrafiltered and spray dried.

Co-precipitate, which is denatured, coagulated milk protein containing casein and whey protein, is typically manufactured by adding calcium chloride to milk and heating indirectly at 80-98° C. in plate heat exchangers or directly by steam injection until coagulation occurs and the product is then dried. Worldwide caseinate manufacture is estimated to be around 250,000 tonnes. Since the casein content of milk is approximately 30 g/L and the BLG content is 4 g/L, if all of the BLG was co-precipitated with the casein, yield would increase by 14%.

The aims of the present invention therefore include:

Devising a more economical method for extracting BLG from whey, yielding not only BLG but low-BLG whey, and turning what would otherwise be a waste product into a source of income.

A new method of casein separation.

A method of modifying casein micelles, to alter their properties.

New components for cheeses, allowing texture, taste etc to be varied.

Preparing a novel food product.

Within this document, references to casein micelles, β-lactoglobulin and other components of milk should be interpreted, unless context requires otherwise, as including all the related inter- and intra-species variants of these components. E.g. β-lactoglobulin will have a slightly different primary structure in different mammals; it will be clear to one skilled in the art that the invention will apply to all these variants.

According to a first aspect of the present invention there is provided a method of isolating β-lactoglobulin from whey comprising the steps of increasing the pH of whey until a pH is reached at which β-lactoglobulin denatures and then decreasing the pH of the resulting mixture until a pH is reached at which β-lactoglobulin precipitates.

Preferably, the pH of whey will be increased by the addition of an alkaline solution and decreased by the addition of an acidic solution.

More preferably, in the step where the pH of whey is increased, the pH will be increased to between 10 and 12.

Preferably also, once the pH has been increased, the whey will be allowed to stand for a period of time.

The period of time will typically be 30 to 120 minutes.

Preferably also, in the step where the pl of the resulting mixtures is decreased, the pH will be reduced to pH 5.

According to a second aspect of the present invention there is provided β-lactoglobulin obtainable by the method of the first aspect.

The denatured β-lactoglobulin may be obtained by the method of the first aspect.

According to a third aspect of the present invention there is provided low β-lactoglobulin content whey obtainable by extracting β-lactoglobulin from the whey according to the method of the first aspect of the present invention.

The low β-lactoglobulin content whey may be obtained by extracting β-lactoglobulin from whey according to the method of the first aspect of the present invention.

According to a fourth aspect of the present invention there is provided a foodstuff having therein β-lactoglobulin prepared according to the first aspect of the present invention.

The foodstuff may consist primarily of β-lactoglobulin prepared according to the first aspect of the present invention and then freeze-dried.

According to a fifth aspect of the present invention there is provided a method of modifying milk having casein micelles, the method comprising the steps of raising the pH of milk until the casein micelles therein are disrupted and subsequently reducing the pH to a value at which the micelles reform.

Preferably, the pH will be raised to between 10 and 12.

More preferably, the pH will be returned to the original pH of the milk.

When c0w's milk is used, the pH may be returned to pH 6.7.

The pH may be returned to a value which increases the quantity of β-lactoglobulin incorporated into cheese by lactose-fermenting micro-organisms.

In these circumstances, the pH will typically be reduced to around 4.6.

According to a sixth aspect of the present invention there is provided a method of modifying casein micelles from milk comprising the steps of adding alkali to milk to raise the pH until the micelles are disrupted, adding or removing chemical constituents and then subsequently adding acid to return the pH to a value at which micelles reform, said chemical constituents being chemicals selected from a group of chemicals which can be incorporated into micelles by this procedure.

Preferably, the pH will be raised to at least 10.

More preferably, the pH will be returned to the original pH of the milk.

When cow's milk is used, the pH may be returned to pH 6.7.

According to a seventh aspect of the present invention there is provided modified milk obtainable by disaggregating and reforming the casein micelles of milk by the method of the fifth or sixth aspect.

Modified milk may be obtained by disaggregating and reforming the casein micelles of milk by the method of the fifth or sixth aspect

According to an eighth aspect of the present invention there is provided dairy produce obtainable by preparing the dairy produce, in an otherwise known method, front milk modified by the method of the fifth or sixth aspect.

Dairy produce may be obtained by preparing, in an otherwise known method, the dairy produce from milk, which has been modified by the method of the fifth or sixth aspect.

According to a ninth aspect of the present invention there is provided a method of coprecipitating β-lactoglobulin and caseinate from milk, the method comprising the steps of increasing the pH of milk, allowing the resulting solution to stand and then reducing the pH of the resulting solution.

The invention will now be described with reference to the following figures in which:

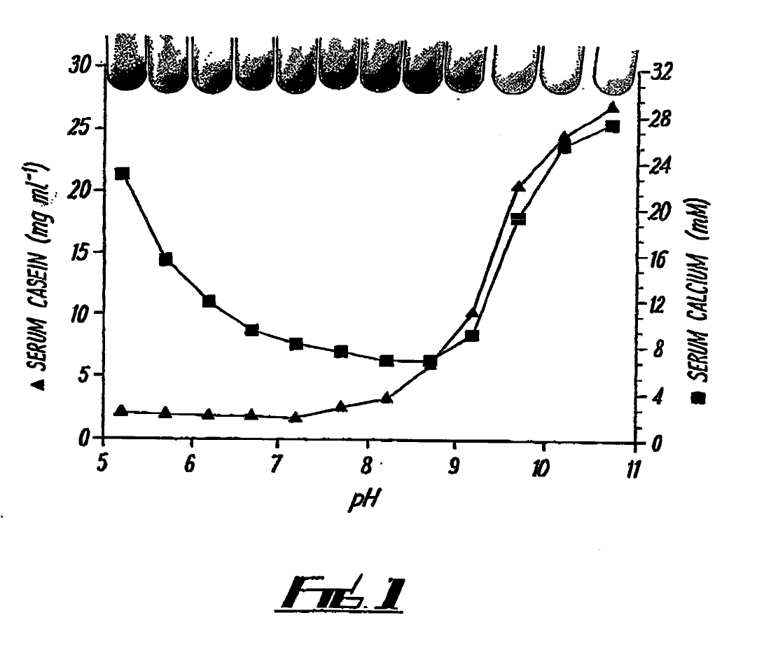

FIG. 1 is a graph showing the soluabilisation of casein protein and calcium as a function of the pH of milk and the change in the volume of the micellar pellet obtained as a result of high speed centrifugation as obtained in Example 1;

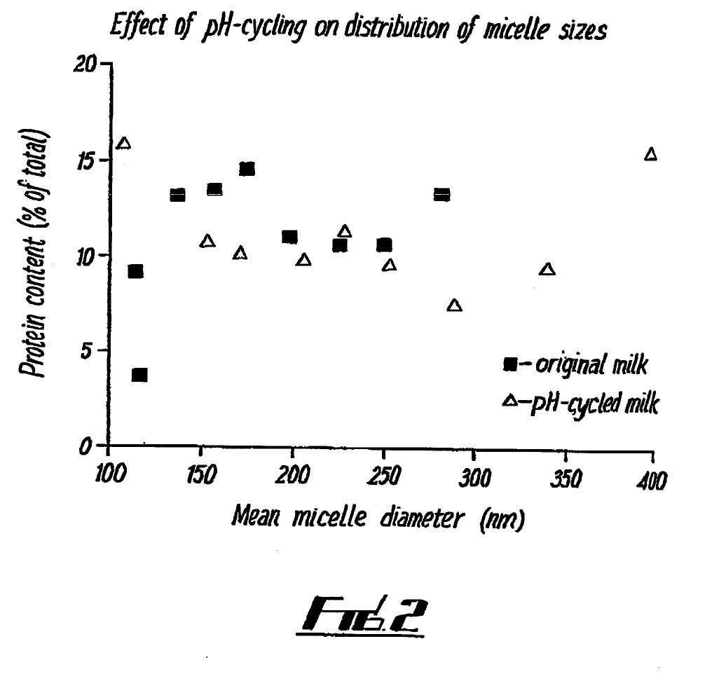

FIG. 2 shows the distribution of casein micellar sizes in the original milk and pH-cycled milk as determined by differential centrifugation in Example 2;

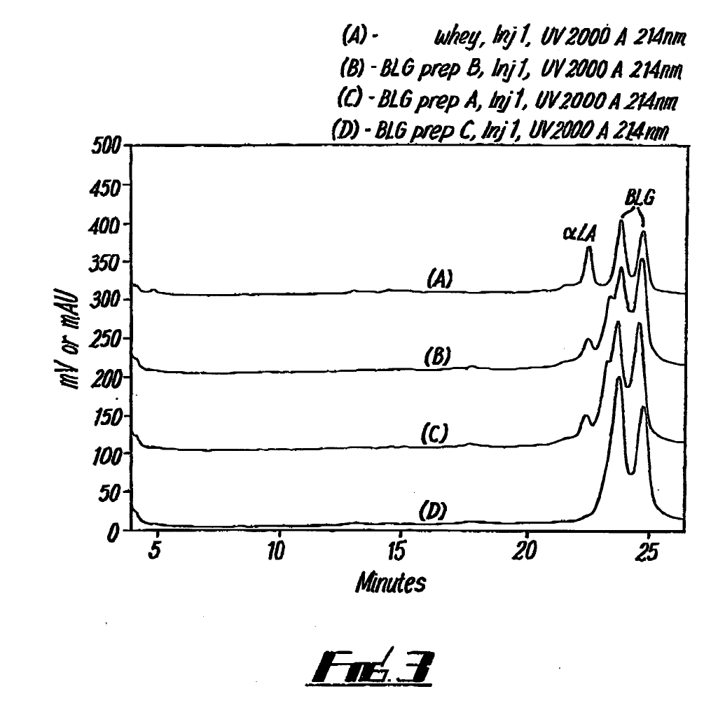

FIG. 3 shows the purity of BLG obtained by pH cycling of cheese whey as detailed in Example 4;

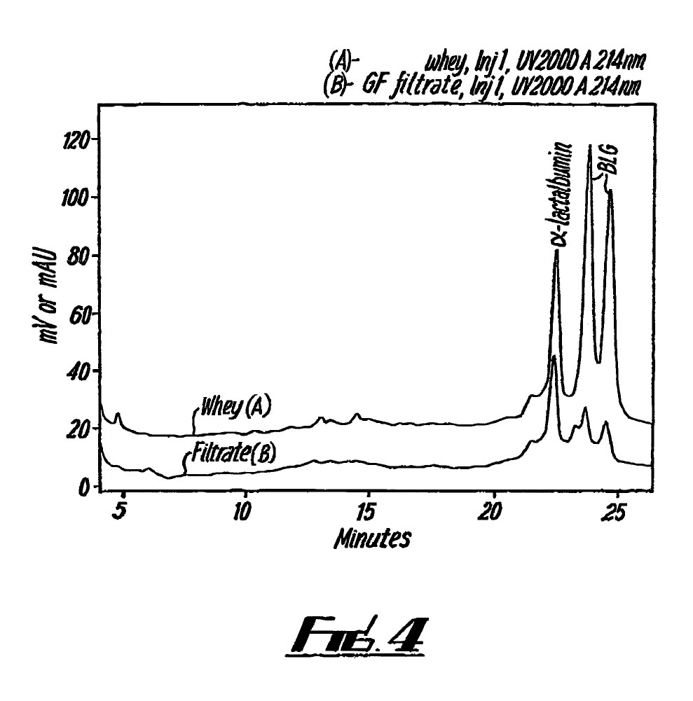

FIG. 4 shows the BLG depleted whey obtained after precipitation of BLG front cheese whey as detailed in Example 4;

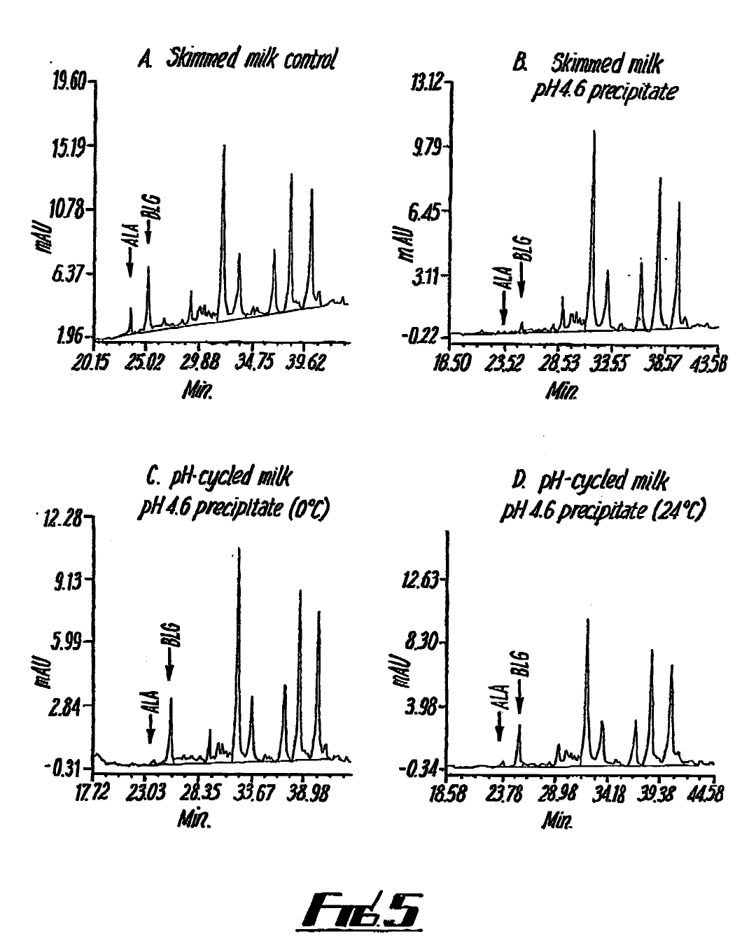

FIG. 5 shows the co-precipitate of casein and BLG obtained after high pH treatment of milk as detailed in example 5;

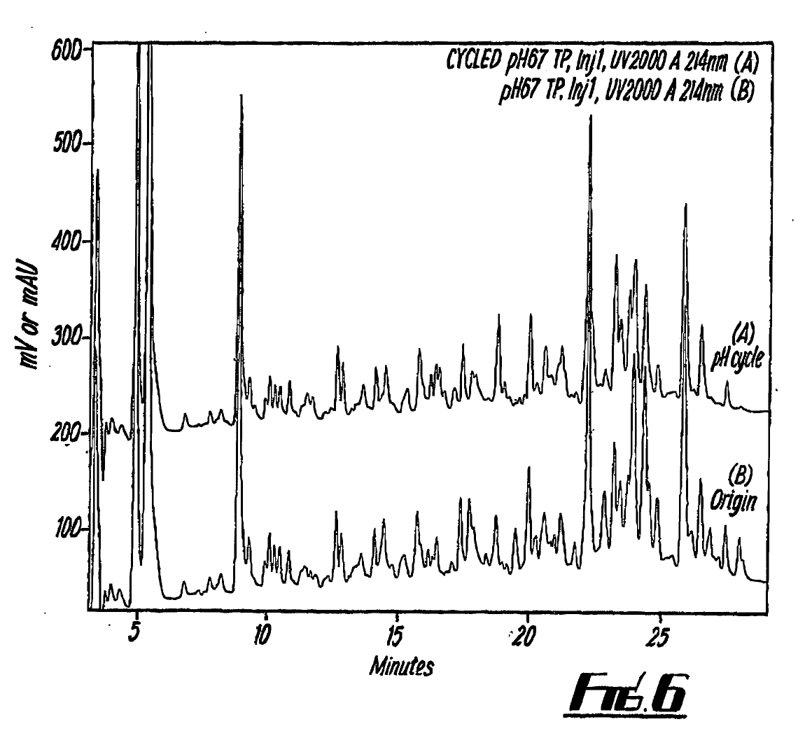

FIG. 6 shows the tryptic peptide map obtained with the original milk and pH-cycled milk to demonstrate the lack of chemical change in the proteins as a result of brief exposure to high pH;

The present invention relates to a low energy method to disaggregate and reform casein micelles in milk and to specifically cause the denaturation and precipitation of a single whey protein, BLG.

There is provided in the present invention a method to cause micelles in milk to disaggregate by raising the pH of the milk to a value greater than 10. This process can be reversed by decreasing the pH of this solution of proteins to that of the original milk. The reformed micelles have different physical characteristics from those of the original micelles. Cheese can still be manufactured from these reformed micelles.

There is also provided a method to specifically purify BLG from cheese whey giving a solid which is very rich in BLG and a solution which is highly depleted.

There is further provided a low energy technique to manufacture a co-precipitate of casein and BLG.

In a first embodiment of the present invention, high and low pH-cycling is used in the manufacture of cheese. FIG. 1 shows the serum concentration of casein and calcium as a function of pH. Photographs of micellar pellets obtained by centrifugation (as described further in Example 1 below) are also shown. This Figure shows that alkaline pH causes disruption of the micelles rendering the caseins soluble and causing the milk to become opalescent since it is scattering of light by the casein micelles which is largely responsible for the whiteness of milk. This may be a useful first step in fractionating the casein component of milk. Individual caseins and specific casein mixtures can potentially be produced from this mixture. This is a reversible process since when the pH is again reduced to 6.7, the natural pH of bovine milk, the micelles again reform but the distribution of micellar sizes is different from the original milk, as evidenced by FIG. 2.

The pH of milk is increased to values between 10 and 12 and the milk is stored for intervals ranging from a few minutes to more than 1 hour to allow the micelles to disaggregate and BLG to denature. The pH is then reduced to 6.7, the natural pH of bovine milk, and the milk is allowed to stand overnight at either room temperature or 4° C. During this period, micelles reform and the milk again appears white. Cheese can then be made as normal by addition of starter micro-organisms and proteolytic enzymes. This pH-cycling influences the gelling behaviour of the milk and possibly also the biochemical changes which occur in cheeses made from milk treated in this way during maturation since the flavour and textural characteristics of the cheese are different from cheese made with untreated milk.

Furthermore, when the casein micelles are reformed, different peptides and other micellar components may be incorporated into the reformed micelles simply by adding them to the solution. This leads to the possibility of preparing milks and cheeses with modified micelles, to give the products different taste, flavour or physical characteristics or to incorporate labels or entirely new components directly or by attaching them to other chemical compounds which will incorporate into the micelles.

It has been known for more than 40 years that at pH 7.5 the protein BLG undergoes a conformational change (JOURNAL OF THE AMERICAN CHEMICAL SOCIETY vol. 81, 1959, pages 4032-4036. Tanford, C., Bunville, L G. and Nozaki, Y. “The reversible trans-formation of β-lactoglobulin at pH 7.5). This has been termed cold denaturation to distinguish it from heat-induced denaturation The method described here utilises this conformational change and subsequent denaturation as a means of specifically precipitating the BLG protein from the whey fraction of milk. Surprisingly, despite nearly all of the proteins present in whey having globular structures which are stabilised in the same manner as that of the BLG molecule, only the BLG molecule undergoes this denaturation and can subsequently be induced to precipitate at acid pH enabling the whey proteins to be fractionated and the BLG to be incorporated into casein aggregates.

In this form of this invention, the whey obtained from cheese-making or caseinate manufacture is adjusted at temperatures between 10 and 38° C. to between pH 10 and 12 by the slow addition of 5M sodium hydroxide. The rate of denaturation of the BLG component in the whey is pH and temperature-dependent being more rapid at higher pH values and lower temperatures. The whey is held at these pH values for 30 to 120 min. The pH is then adjusted to pH 5 by the slow addition of either 5M hydrochloric acid or lactic acid. The whey is then allowed to stand without stirring. A heavy precipitate forms which on prolonged standing flocculates and settles at the bottom of the vessel. Most of the supernatant layer can then be removed by decanting and the flocculated protein in the remaining lower level is harvested by centrifugation. This material is washed by resuspending in a small volume of water at pH 5 and recentrifuging. The solid material is then freeze-dried.

Analysis of the freeze-dried solid by reverse phase high performance shows it to be almost pure BLG, the percentage, purity varying between 90 and 100% (FIGS. 3). Typical yields of solid are 3 to 4 g/L of whey and more than 70% of the total BLG can be extracted. The supernatant from the process was shown to be correspondingly depleted of BLG (FIG. 4) and may itself have different functional properties and uses from the starting whey. For example, human milk does not contain BLG and indeed this is known to be the protein in cow's milk that is most likely to cause allergenic reactions in infants. Whey protein isolates having low levels of BLG should be of interest to infant formula manufacturers since the proportion of the other proteins will be correspondingly increased.

The BLG fraction is largely insoluble but forms a smooth paste with good mouth-feel, flavour and aroma. It may therefore be useful as a fat substitute in cheeses and other processed foods. The BLG-depleted soluble fraction can subsequently be processed further either by concentration and drying or fractionated to give other protein fractions. By virtue of being depleted of BLG the soluble fraction is enriched in the other whey proteins and may be of interest in the manufacture of infant formulations.

It was found that freeze-dried BLG prepared according to this method, when mixed with water, gave a tasty toffee flavoured spread. This leads to the potential application of BLG, particularly the form prepared by this process, by itself or in a mixture with other edible materials, as a tasty, high protein foodstuff.

pH-cycling may also be used to increase protein recovery in the manufacture of cottage cheese. The ph of reformed pH-cycled milk is reduced to 4.6 by the addition of either acid or lactose-fermenting micro-organisms. Due to cold-denaturation of the BLG, this protein should be incorporated into the cheese curd rather than being lost in the waste whey increasing the protein recovery and reducing the protein present in the waste from the process and hence the BOD of the waste

In a further process utilising pH-cycling, a co-precipitate of BLG and caseinate which may also have interesting functional properties can be produced. The pH of skimmed milk is raised to 11 by addition of sodium hydroxide. After a minimum of 1 hour the pH is reduced to 4.6 by addition of mineral acid. The casein, together with the denatured BLG, co-precipitate leaving an acid whey which contains little BLG. As much as 90% of the BLG can be removed from the acid whey and incorporated into the co-precipitate (FIG. 5).

In addition to inducing denaturation of BLG, the high pH used in these methods also partially sterilises the milk and wheys used in the manufacture of all of these products reducing the growth of microorganisms and possibly removing the need for a heat-sterilisation step.

Short exposure to high pH sufficient to induce the changes reported here, does not appear to cause chemical damage to the milk proteins (FIG. 6).

### EXAMPLE 1

The pH of skim milk was adjusted to values between 5.2 and 10.7 by the slow addition of either 1M NaOH or 1M HCl. After incubating for 1 hour at room temperature, the sample was centrifuged and the supernatant removed. The amount of casein in the supernatant fractions wan determined by high performance liquid chromatography in the reverse phase mode (RP-HPLC). The calcium content of the supernatant phase was determined using a calorimetric assay. The results (FIG. 1) show that at acid pH values, below the natural pH of milk, a small amount of casein was present in the supernatant phase. The amount of soluble calcium increased as the pH reduced. Above pH 6.7 the amount of soluble casein increased until at pH 10.7 a level of 28 mg of casein per ml of milk was soluble which is equivalent to all of the casein in the milk. As the pH increased the size of the pellet in the centrifuge tubes initially increased up to around pH 8.3 as the micelles became more swollen and then decreased. Similarly, the milk became increasingly less white in appearance and at the highest pH was translucent and green/brown in colour. The amount of calcium in the supernatant phase also increased with pH. High pH is therefore a simple method to disrupt casein micelles and solubilise all of the casein and calcium. This may be a useful solubilisation step prior to fractionating individual casein proteins.

### EXAMPLE 2

The pH of skimmed milk was increased to 12.0 by the addition at room temperature, with stirring, of 5M NaOH. Immediately that the milk lost its whiteness, the pH was readjusted to 6.7, the natural pH of bovine milk, by the slow addition, with stirring at room temperature, of 5M HCl. Any flocs which formed during the addition of HCl were transitory in nature and soot dissolved. After incubating for 2 hours at pH 6.7, the pH-cycled milk together with a sample of the original milk, was subjected to a series of centrifugation steps of increasing duration/severity in order to fractionate the casein micelles largely on the basis of their size. This is termed differential centrifugation. The size of the micelles in each of the pellet fractions and in the final supernatant fraction was measured by photon correlation spectroscopy after resuspension of the pellets at the appropriate concentration in milk ultrafiltrate. The protein content of these individual fractions was determined by RP-HPLC. The results are shown in FIG. 2. The size distribution of the micelles disaggregated and reformed by pH-cycling were significantly different from those in the original milk, this may be useful as a means of changing the texture of products such as cheese and yogurt where micellar diameter is important.

### EXAMPLE 3

The pH of two, 45 litre vats of milk was adjusted to 10.5 by the addition of 1M NaOH. After 2 hours, the pH of one vat was reduced to 6.7. Both vats were stored at 4° C. for 16 hours. The pH of the second vat was then adjusted to 6.7. Both vats were then incubated at 37° C. for 2 hours. In both cases, the micelles dissociated and reformed as above (FIG. 3). Cloned chymosin and starter culture micro-organisms were then added to both vats in the normal Cheddar cheese manufacturing process. The pH-cycled milks formed a rennet gel, the time required to do so being slightly longer than that required for an untreated milk. This gel was slightly less firm that that obtained from an untreated milk but could be scalded and salted in the usual process. The Cheddar cheese so formed was allowed to mature at low temperature and was sampled at intervals for flavour and texture. Both of these aspects were good and were different from that obtained with cheese made from untreated milk using the process. pH-Cycling of milk offers a way to change the flavour and textural characteristics of cheese.

### EXAMPLE 4

The pH of three 10 litre batches of sweet whey, the waste liquor from the manufacture of cheese was adjusted to pH 11 in cases A and B and pH 10 in case C. After storing A at room temperature for 2 hours, the pH was adjusted to 5.1. In case B the pH was adjusted to 7.5 after 2 hours storage at room temperature. After a further 2 hours at this pH, the pH was adjusted to 5.1. In case C, after 2 hours at pH 10.0, the pH was reduced to 5.1. In all cases the whey was stored overnight at pH 5.1 and 4 C. A heavy flocculate quickly began to form which then formed a precipitate on the bottom of the container. Much of the supernatant was removed by decantation and the precipitate was finally recovered by centrifugation and was washed by resuspending in an equal volume of water adjusted to pH 5. The precipitates were then freeze-dried. A sample of the whey after removal of the precipitated material was retained for analysis. The freeze-dried powders proved to be largely insoluble in water at any pH. However, they did form very smooth pastes some of which had an interesting, creamy flavour. Analysis of these powders by RP-HPLC after dissolving them in a buffer consisting of 7M urea and 60 mM 2-mercaptoethanol at pH 7 showed them to be almost pure BLG (FIG. 4). The degree of purity was estimated to be greater than 90%, the major impurity being trace amounts of α-lactalbumin. The solubility in this buffer in contrast to the insolubility in water indicates that the insolubility is due to the individual BLG molecules forming oligomers and polymers linked via disulphide bridges as a result of the pH treatment. Typically, more than 70% of the BLG content of the whey could be recovered by this technique. The insoluble BLG nay be useful as a fat replacer or for inclusion in cheese curd in order to improve the texture of low-fat cheese.

The whey remaining after removal of the insoluble BLG was shown to be correspondingly enriched in α-lactalbumin and to contain relatively little BLG (FIG. 5). This may be useful in the manufacture of infant formula since human milk is high in α-lactalbumin and contains no BLG which is the major cause of allergic response to bovine milk proteins in infants.

### EXAMPLE 5

One 20 ml batch of skimmed bovine milk was adjusted to pH11 at 0° C. and a similar batch was adjusted to this pH at 24° C. After 10 minutes the pH of both was adjusted to 4.6 by the addition of 1M HCl. This caused a precipitate to form as in the manufacture of acid caseinate. Samples of the insoluble material and of the insoluble material produced from milk which had not been subjected to the pH 11 treatment were analysed by capillary electrophoresis (FIG. 6). The results show that whereas in the normal milk, the protein in the precipitate contained only trace amounts of α-lactalbumin and BLG, the precipitate from the milks subjected to the pull step contained almost all of the BLG present in the milk, but only trace amounts of α-lactalbumin. This specific co-precipitate of BLG with casein would increase the amount of protein which can be produced by traditional caseinate manufacture by approximately 15%. The material so formed may have interesting processing properties and the whey produced as a by-product may also be of interest in the manufacture of other products such as infant formula.

### EXAMPLE

In order to ensure that alkaline treatment did not cause possibly harmful changes in the proteins, a sample of original and pH cycled milk was subject to hydrolysis by trypsin. The tryptic peptides formed by proteolytic action were analysed by RP-HPLC (FIG. 7) The peptide patterns so foxed were very similar indicating that there was no significant changes in the chemical properties of the proteins as a result of the pH-cycling.

Further modifications and improvements may be incorporated without departing form the scope of the invention herein intended.

1. A method of isolating β-lactoglobulin from whey comprising the steps of increasing the pH of whey until a pH is reached at which β-lactoglobulin denatures and then decreasing the pH of the resulting mixture until a pH is reached at which β-lactoglobulin precipitates.

2. A method of isolating β-lactoglobulin from whey as claimed in claim 1 wherein the pH of whey is increased by the addition of an alkaline solution and decreased by the addition of an acidic solution.

3. A method of isolating β-lactoglobulin from whey as claimed in any one of the preceding claims wherein the pH of whey is increased to between 10 and 12.

4. A method of isolating β-lactoglobulin from whey as claimed in any one of the preceding claims wherein once the pH has been increased, the whey is allowed to stand for a period of time.

5. A method of isolating β-lactoglobulin from whey as claimed in claim 4 wherein the period of time is 30 to 120 minutes.

6. A method of isolating β-lactoglobulin from whey as claimed in any one of the preceding claims wherein the pH of the resulting mixtures is decreased to pH 5.

7. β-lactoglobulin obtainable by the method of any of claims 1-6.

8. β-lactoglobulin as claimed in claim 7 obtained by the method of any of claims 1-6.

9. Low β-lactoglobulin content whey obtainable by the method of any of claims 1-6.

10. Low β-lactoglobulin content whey as claimed in claim 9 obtained by the method of any of claims 1-6.

11. A foodstuff containing β-lactoglobulin wherein the, β-lactoglobulin is prepared by the method of any of claims 1-6.

12. A foodstuff as claimed in claim 11 wherein the foodstuff consists primarily of β-lactoglobulin prepared by the method of any of claims 1-6 and freeze dried.

13. A method of modifying milk having casein micelles, the method comprising the steps of raising the pH of milk until the casein micelles therein are disrupted and subsequently reducing the pH to a value at which the micelles reform.

14. A method of modifying milk having casein micelles as claimed in claim 13 wherein the pH is raised to between 10 and 12.

15. A method of modifying milk having casein micelles as claimed in claims 13-14 wherein the pH is reduced to the original pH of the milk.

16. A method of modifying milk having casein micelles as claimed in claims 13-15 wherein the pH is reduced to a value which increases the quantity of β-lactoglobulin incorporated into cheese by lactose-fermenting micro-organisms.

17. A method of modifying milk having casein micelles as claimed in claims 13-16 wherein the pH is reduced to around 4.6.

18. Modified milk obtainable by disaggregating and reforming the casein micelles of milk by the method described in any of the claims 13-17.

19. Modified milk as claimed in claim 18 obtained by the method of any of claims 13-17.

20. Dairy produce obtainable by preparation, in an otherwise known method, from milk modified by the method of any of claims 13-17.

21. A method of modifying casein micelles from milk comprising the steps of adding alkali to milk to raise the pH until the micelles are disrupted, adding or removing chemical constituents and then subsequently adding acid to return the pH to a value at which micelles reform, said chemical constituents being chemicals selected from a group of chemicals which can be incorporated into micelles by this procedure.

22. A method of modifying casein micelles from milk as claimed in claim 20 wherein the pH is raised to at least 10.

23. A method of modifying casein micelles from milk as claimed in claims 20-21 wherein the pH is returned to the original pH of the milk.

24. Modified milk obtainable by disaggregating and reforming the casein micelles of milk by the method described in any of claims 20-22.

25. Modified milk as claimed in claim 23 obtained by the method of any of claims 20-22.

26. Dairy produce obtainable by preparation, in an otherwise known method, from milk modified by the method of any of claims 20-22.

27. A method of coprecipitating β-lactoglobulin and caseinate from milk, the method comprising the steps of increasing the pH of milk, allowing the resulting solution to stand and then reducing the pH of the resulting solution.
